(19) United States
(12) Patent Application Publication
(10) Pub. No.: US 2013/0252876 A1
(43) Pub. Date: Sep. 26, 2013

(54) COMPOSITIONS AND METHOD FOR PROMOTING MUSCULOSKELETAL REPAIR

(71) Applicant: Marc S. Penn, Beachwood, OH (US)

(72) Inventor: Marc S. Penn, Beachwood, OH (US)

(73) Assignee: The Cleveland Clinic Foundation, Clevelantd, OH (US)

(21) Appl. No.: 13/823,132

(22) PCT Filed: Sep. 13, 2011
(86) PCT No.: PCT/US11/51340

§ 371 Date: Jun. 13, 2013

Related U.S. Application Data
(60) Provisional application No. 61/383,035, filed on Sep. 15, 2010.

(51) Int. Cl. A61K 38/19; A61F 2/28

(52) U.S. Cl. CPC A61K 38/195; A61F 2/28
USPC 514/1.1; 623/23.61

(57) ABSTRACT

A method of treating a musculoskeletal injury in a subject includes administering directly to a site of the musculoskeletal injury or to an area proximate the musculoskeletal injury an amount of SDF-1, MCP-3, or combinations thereof effective to promote repair of the musculoskeletal injury of the subject and recruit connective tissue progenitor cells to the site of the musculoskeletal injury.

### RELATED APPLICATION

This application claims priority from U.S. Provisional Application No. 61/383,035, filed Sep. 15, 2010 and Ser. No. 12/808,056 filed Jun. 14, 2010, the subject matter of which are incorporated herein by reference in their entirety.

### TECHNICAL FIELD

This application relates to methods and compositions for promoting musculoskeletal repair in a subject.

### BACKGROUND OF THE INVENTION

Approximately 5% to about 10% of human fractures in the United States fail to heal in a timely manner. Orthopedic treatments for delayed unions and non-unions are as varied as the risk factors. Essential steps in fracture healing are the recruitment, proliferation, and differentiation of marrow stromal cells (MSCs) into chondrocytes and osteoblasts. Most studies for stem-cell therapy in fracture healing have focused on the direct engraftment of stem cells into the local site or systemic circulation. Another possible source of stem cells is osteogenic cells present in the peripheral circulation.

One possible signaling pathway to recruit progenitors from the systemic circulation, stem cell niches throughout the body or endogenous cells within the organ or tissue involves stromal-derived factor-1 (SDF-1), a CXC chemokine, and CXC receptor 4 (CXCR4). This pathway is required for stem cell retention within the bone marrow and is being applied in clinical stem cell harvest and transplantation procedures. Moreover, local expression of SDF-1 has been shown to recruit hematopoietic/endothelial progenitor cells to ischemic sites.

Another possible signaling pathway to recruit progenitors from the systemic circulation, stem cell niches throughout the body or endogenous cells within the organ or tissue involves monocyte chemotactic protein 3 (MCP-3). As a member of the cysteine-cysteine (CC) chemokine family, MCP-3 is a key mediator of pro-inflammatory pathways, activating all leukocytes by binding to several chemokine receptors. Recent work has identified MCP-3 as a homing factor for MSCs.

### SUMMARY OF THE INVENTION

This application relates to a method of treating a musculoskeletal injury in a subject. The method includes administering directly to the musculoskeletal injury or to an area proximate the musculoskeletal injury an amount of SDF-1, MCP-3, and/or combinations thereof effective to promote healing of the musculoskeletal injury of the subject. The SDF-1, MCP-3, and/or combinations thereof can also be administered at an amount effective to increase homing of connective tissue progenitor cells to the skeletal injury site. In some aspects, the SDF-1, MCP-3, and/or combinations thereof can be administered at an amount effective to induce differentiation of osteogenic progenitor cells to osteoblasts and/or promote osteogenesis at the site of the musculoskeletal injury.

This application also relates to a method of treating a skeletal fracture in a subject. The method includes administering directly to the skeletal fracture or to an area proximate the skeletal fracture an amount of SDF-1, MCP-3, and/or combinations thereof effective to promote healing of the skeletal fracture. The SDF-1, MCP-3, and/or combinations thereof can also be administered at an amount effective to increase homing of connective tissue progenitor cells to the skeletal fracture. In some aspects, the SDF-1, MCP-3, and/or combinations thereof can be administered at an amount effective to induce differentiation of osteogenic progenitor cells to osteoblasts and/or promote osteogenesis at the site of the skeletal fracture.

This application further relates to a bone graft or bone graft substitute for treating a musculoskeletal injury. The bone graft or bone graft substitute includes an osteoconductive matrix and an amount of SDF-1, MCP-3, or combinations thereof effective to promote repair of the musculoskeletal injury of the subject and recruit connective tissue progenitor cells to the site of the musculoskeletal injury. In some aspect, the bone graft or bone graft substitute can include a population of cells. The cells can over express the amount of SDF-1 and/or MCP-3 effective to promote repair of the skeletal injury of the subject and recruit connective tissue progenitor cells to the site of the musculoskeletal injury. In some aspects, the SDF-1 and/or MCP-3 can be expressed at an amount effective to induce differentiation of osteogenic progenitor cells to osteoblast and/or promote osteogenesis at the site of the musculoskeletal injury. In some aspects, the bone graft further includes an osteoconductive matrix.

This application further relates to cell delivery for treating a musculoskeletal injury. The cell population can include autologous or allogeneic mesenchymal stem cells with or without other stem cell or cell populations and an amount of SDF-1, MCP-3, or combinations thereof effective to promote repair of the musculoskeletal injury of the subject and enhance survival of the delivered cells or recruit connective tissue progenitor cells to the site of the musculoskeletal injury. The cells can over express the amount of SDF-1 and/or MCP-3 effective to promote repair of the skeletal injury of the subject and enhance their survival and/or recruit connective tissue progenitor cells to the site of the musculoskeletal injury. In some aspects, the SDF-1 and/or MCP-3 can be expressed at an amount effective to induce differentiation of osteogenic progenitor cells to osteoblast and/or promote osteogenesis at the site of the musculoskeletal injury.

### BRIEF DESCRIPTION OF THE DRAWINGS

FIG. 1 illustrates a schematic drawing showing time course of parabiosis, fracture and harvest procedures. Fibular osteotomy was performed four weeks after parabiosis. Mice were euthanized two weeks post osteotomy.

FIG. 2 illustrates images showing histological findings of fracture callus in parabiosis wild-type partner. A. Histological image of fracture site (10×).

FIG. 3 is a chart showing the percentage of total cells within the fracture callus that express GFP. Roughly 80% GFP⁺ expressing cells were also AP⁺. Both SDF-1 and MCP-3 secreting scaffolds resulted in statistically significant increases in GFP⁺ cell recruitment relative to MSCs alone.

FIG. 4 is a chart showing the percentage of total AP+ cells within the fracture callus that participated in fracture repair at two weeks. There was no significant difference in activity amongst all five groups.

FIG. 5 is a chart showing the percentage of total cells within the fracture callus that are positive for both GFP and AP. Both SDF-1 and MCP-3 secreting of MSCs scaffolds resulted in statistically significant increases in GFP⁺/AP⁺ cell recruitment relative to MSCs alone.

FIG. 6 is a chart showing the cell density within the fracture callus. * indicates a significant difference (p<0.025) as compared to treatment with MSCs alone.

### DETAILED DESCRIPTION

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers. Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992. Conventional methods of gene transfer and gene therapy can also be adapted for use in the present invention. See, e.g., Gene Therapy: Principles and Applications, ed. T. Blackenstein, Springer Verlag, 1999; Gene Therapy Protocols (Methods in Molecular Medicine), ed. P. D. Robbins, Humana Press, 1997; and Retro-vectors for Human Gene Therapy, ed. C. P. Hodgson, Springer Verlag, 1996.

As used herein, the term “promoting musculoskeletal repair” or “promoting healing of a skeletal injury” means augmenting, accelerating, improving, increasing, or inducing closure, healing, or repair of a skeletal injury.

As used herein, the terms “treating” and “treatment” refer to the improvement or remediation of damage, and the reduction in severity and/or frequency of symptoms, elimination of symptoms and/or underlying cause, prevention of the occurrence of symptoms and/or their underlying cause. Thus, for example, “treating” of a skeletal injury includes increasing healing at a skeletal injury site. Thus, for example, the present method of “treating” a subject in need of skeletal fracture therapy encompasses the treatment of a skeletal injury site that is in need of healing.

As used herein, the term “connective tissue progenitor cell” refers to any cell which, when exposed to appropriate stimuli, may differentiate and/or become capable of producing, secreting components, and/or displaying a phenotype characteristic of connective tissue, such as skeletal tissue.

As used herein, the term “osteogenic cell”, “osteoprogenitor precursor” or “osteogenic progenitor cell” refers to any cell which, when exposed to appropriate stimuli, may differentiate and/or become capable of producing, secreting components, and/or displaying a phenotype characteristic of bone tissue or cells capable of bone formation.

As used herein, the term “osteoconductive” refers to any structure or material that facilitates the formation of bone structure. For example, a structure or material with the ability to serve as a scaffold on which bone cells can attach, migrate, grow and divide.

In the context of the present invention, the term “population” refers to an isolated culture comprising a homogenous, a substantially homogenous, or a heterogeneous culture of cells. Generally, a “population” may also be regarded as an “isolated” culture of cells.

This application relates to compositions and method that can be used for the treatment of a musculoskeletal injury and/or the promotion of musculoskeletal repair. The musculoskeletal injury can be treated by administering to the site the musculoskeletal injury or to an area proximate the musculoskeletal injury of the subject a therapeutically effective amount of a signaling molecule (e.g., stromal cell-derived factor-1 (SDF-1) and/or monocyte chemotactic protein-3 (MCP-3)). The methods and compositions described herein contemplate that administration of SDF-1 and/or MCP-3 to a skeletal injury or to an area proximate the skeletal injury can facilitate recruitment of connective tissue cells and progenitor cells, such as connective tissue progenitor cells (e.g., osteogenic progenitor cells), from systemic circulation to the site of the musculoskeletal injury to facilitate musculoskeletal repair, induce differentiation of osteogenic progenitor cells to osteoblasts and/or promote osteogenesis at the site of the musculoskeletal injury.

It was found that the signaling molecules SDF-1 and MCP-3 can enhance homing of connective tissue progenitor cells (CTPs) that retain the capability of bone formation into sites of musculoskeletal injury. SDF-1 and MCP-3 participate in osteogenic progenitor cell recruitment. Osteogenic progenitor cells that home to a site of musculoskeletal injury by SDF-1 and/or MCP-3 recruitment can be induced to differentiate into a wide variety of connective tissue-specific cells, including chondrocytes and osteoblasts. Connective tissue progenitor cells can differentiate into specialized and/or partially specialized cells, which can repopulate (i.e., engraft) and partially or wholly restore the normal function of the musculoskeletal tissue being treated.

One aspect of the application therefore relates to a method of treating a musculoskeletal injury in a subject by administering SDF-1 and/or MCP-3 directly to a skeletal injury or to an area proximate the skeletal injury. The SDF-1 and/or MCP-3 administered can be at an amount effective to promote healing of a musculoskeletal injury of the subject. In some aspects, the amount of SDF-1 and/or MCP-3 administered to the subject is an amount effective to increase homing of CTPs to the musculoskeletal injury site or to an area proximate the musculoskeletal injury site. In other aspects, the SDF-1 and/or MCP-3 can be administered at an amount effective to induce differentiation of osteogenic progenitor cells to osteoblasts and/or promote osteogenesis at the site of the musculoskeletal injury.

A musculoskeletal injury, or a skeletal injury, as contemplated by the application can include any injury to any portion of the musculoskeletal system (e.g., bone) of a subject. Examples of musculoskeletal injuries include damage to skeletal tissues, such as bone fracture, injuries sustained during medical procedures, such as bone grafting; trauma-induced injuries, such as cuts, incisions, injuries sustained as result of accidents, post-surgical injuries, tumor or cancer related musculoskeletal injuries, and injuries following dental surgery. Such musculoskeletal injuries can also be the direct or indirect result of an external force, with or without disruption of structural continuity.

It will be appreciated that the present application is not limited to the preceding skeletal injuries and that other injuries can be treated by the compositions and methods of the present invention.

Mammalian subjects, which will be treated by methods and compositions of the present invention, can include any mammal, such as human beings, rats, mice, cats, dogs, goats, sheep, horses, monkeys, apes, rabbits, cattle, etc. The mammalian subject can be in any stage of development including adults, young animals, and neonates. Mammalian subjects can also include those in a fetal stage of development.

In some aspects of the application, the MCP-3 can be administered to the musculoskeletal injury or an area proximate the musculoskeletal injury before, after, or at substantially the same time as the administration of the SDF-1.

The duration of time that the SDF-1 and/or MCP-3 is administered directly to the skeletal injury or to an area proximate the injury can comprise from about onset of the skeletal injury to about days, weeks, or months after the skeletal injury.

One example of a particular type of connective tissue progenitor cell (CTPs) that can be homed or recruited to a musculoskeletal injury site by the SDF-1 and/or MCP-3 in accordance with the application is a mesenchymal stem cell (MSC). MSCs include the formative pluripotent blast or embryonic cells that differentiate into the specific types of connective tissues, (i.e., the tissue of the body that support specialized elements, particularly including adipose, osseous, cartilaginous, elastic, muscular, and fibrous connective tissues depending on various in vivo or in vitro environmental influences). Another example of a CTP that can be homed or recruited to a skeletal injury site by SDF-1 and/or MCP-3 is a multipotent adult progenitor cell (MAPC) (e.g., skeletal derived MAPC). MAPCs in accordance with the present invention comprise adult progenitor or stem cells that are capable of differentiating into cells types beyond those of the tissues in which they normally reside (i.e., exhibit plasticity). Still other examples of a CTP that can be homed or recruited to a skeletal injury site by SDF-1 and/or MCP-3 can include osteogenic progenitor cells that are capable of differentiating to osteoblasts and osteocytes.

In aspect of the application, SDF-1 that is administered directly to a musculoskeletal injury or to an area proximate the musculoskeletal injury can have an amino acid sequence that is substantially similar to a native mammalian SDF-1 amino acid sequence. The amino acid sequence of a number of different mammalian SDF-1 protein are known including human, mouse, and rat. The human and rat SDF-1 amino acid sequences are about 92% identical. SDF-1 can comprise two isoform, SDF-1 alpha and SDF-1 beta, both of which are referred to herein as SDF-1 unless identified otherwise.

The SDF-1 can have an amino acid sequence substantially identical to one of the foregoing mammalian SDF-1 proteins. For example, the SDF-1 that is over-expressed can have an amino acid sequence substantially similar to SEQ ID NO: 1. SEQ ID NO: 1 is the amino sequence for human SDF-1 and is identified by GenBank Accession No. NP954637. The SDF-1 that is over-expressed can also have an amino acid sequence that is substantially identical to SEQ ID NO: 2. SEQ ID NO: 2, includes the amino acid sequences for rat SDF and is identified by GenBank Accession No. AAF01066.

SDF-1 in accordance with the application can also be a variant of mammalian SDF-1, such as a fragment, analog and derivative of mammalian SDF-1. Such variants include, for example, a polypeptide encoded by a naturally occurring allelic variant of native SDF-1 gene (i.e., a naturally occurring nucleic acid that encodes a naturally occurring mammalian SDF-1 polypeptide), a polypeptide encoded by an alternative splice form of a native SDF-1 gene, a polypeptide encoded by a homolog or ortholog of a native SDF-1 gene, and a polypeptide encoded by a non-naturally occurring variant of a native SDF-1 gene.

SDF-1 variants have a peptide sequence that differs from a native SDF-1 polypeptide in one or more amino acids. The peptide sequence of such variants can feature a deletion, addition, or substitution of one or more amino acids of a SDF-1 variant Amino acid insertions are preferably of about 1 to 4 contiguous amino acids, and deletions are preferably of about 1 to 10 contiguous amino acids. Variant SDF-1 polypeptides substantially maintain a native SDF-1 functional activity. Examples of SDF-1 polypeptide variants can be made by expressing nucleic acid molecules within the invention that feature silent or conservative changes.

SDF-1 polypeptide fragments corresponding to one or more particular motifs and/or domains or to arbitrary sizes, are within the scope of the present invention. Isolated peptidyl portions of SDF-1 can be obtained by screening peptides recombinantly produced from the corresponding fragment of the nucleic acid encoding such peptides. For example, a SDF-1 polypeptides of the present invention may be arbitrarily divided into fragments of desired length with no overlap of the fragments, or preferably divided into overlapping fragments of a desired length. The fragments can be produced recombinantly and tested to identify those peptidyl fragments, which can function as a signaling molecule capable of increasing homing of CTPs to a site of a skeletal injury.

Variants of SDF-1 polypeptides can also include recombinant forms of the SDF-1 polypeptides. Recombinant polypeptides preferred by the present invention, in addition to SDF-1 polypeptides, are encoded by a nucleic acid that can have at least 70% sequence identity with the nucleic acid sequence of a gene encoding a mammalian SDF-1.

SDF-1 variants can include forms of the protein that constitutively express the functional activities of native SDF-1. Other SDF-1 variants can include those that are resistant to proteolytic cleavage, as for example, due to mutations, which alter protease target sequences. Whether a change in the amino acid sequence of a peptide results in a variant having one or more functional activities of a native SDF-1 can be readily determined by testing the variant for a native SDF-1 functional activity.

In another aspect of the application, MCP-3 that is administered to a musculoskeletal injury or an area proximate the musculoskeletal injury to promote healing of the skeletal injury of a subject can have an amino sequence substantially similar to native mammalian MCP-3. For example, the MCP-3 can have amino sequences substantially similar to, respectively, SEQ ID NO: 5, which is substantially similar to the nucleic sequences of, respectively, GenBank Accession No. CAA50407.

MCP-3 can also be a variant of native MCP-3, such as a fragment, analog and derivative of mammalian MCP-3. Such variants can include, for example, a polypeptide encoded by a naturally occurring allelic variant of a native MCP-3 gene (i.e., a naturally occurring nucleic acid that encodes a naturally occurring mammalian MCP-3), a polypeptide encoded by an alternative splice form of a native MCP-3 gene, a polypeptide encoded by a homolog or ortholog of a native MCP-3 gene, and a polypeptide encoded by a non-naturally occurring variant of a native MCP-3 gene.

MCP-3 variants can have a peptide (or amino acid) sequence that differs from native MCP-3 in one or more amino acids. The peptide sequence of such variants can feature a deletion, addition, or substitution of one or more amino acids of MCP-3 protein Amino acid insertions are preferably of about 1 to 4 contiguous amino acids, and deletions are preferably of about 1 to 10 contiguous amino acids. Variant MCP-3 proteins substantially maintain a native MCP-3 protein functional activity. Examples of MCP-3 protein variants can be made by expressing nucleic acid molecules within the invention that feature silent or conservative changes.

MCP-3 protein fragments corresponding to one or more particular motifs and/or domains or to arbitrary sizes, are within the scope of the present invention. Isolated peptidyl portions of MCP-3 proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, a MCP-3 protein of the present invention may be arbitrarily divided into fragments of desired length with no overlap of the fragments, or preferably divided into overlapping fragments of a desired length. The fragments can be produced recombinantly and tested to identify those peptidyl fragments which can function as agonists of a native MCP-3 protein.

Variants of MCP-3 protein can also include recombinant forms of the proteins. Recombinant polypeptides preferred by the present invention, in addition to a MCP-3 protein, are encoded by a nucleic acid that can have at least 70% sequence identity with the nucleic acid sequence of a gene encoding a mammalian protein.

MCP-3 protein variants can include forms of the protein that constitutively express the functional activities of a native MCP-3 protein. Other protein variants can include those that are resistant to proteolytic cleavage, as for example, due to mutations, which alter protease target sequences. Whether a change in the amino acid sequence of a peptide results in a variant having one or more functional activities of a native MCP-3 protein can be readily determined by testing the variant for a native MCP-3 protein functional activity.

In another aspect, the SDF-1 and/or MCP-3 can be administered directly to a a site of musculoskeletal injury or to an area proximate the injury by introducing an agent into cells proximate the muscloskeletal injury that causes, increases, and/or upregulates expression of SDF-1 and/or MCP-3 in cells proximate the skeletal injury or about the periphery of the skeletal injury. SDF-1 and/or MCP-3 protein expressed in cells proximate the skeletal injury can be an expression product of a genetically modified cell. Where the SDF-1 and MCP-3 are expressed from a target cell proximate the musculoskeletal injury at substantially the same time, the target cell can be transfected with a bicistronic expression construct that expresses the SDF-1 and MCP-3. Bicistronic expression constructs are known in the art and can be readily employed in the present therapeutic process.

The target cells can include cells within or about the periphery of the musculoskeletal injury or ex vivo cells that are biocompatible with musculoskeletal tissue being treated. The biocompatible cells can also include autologous cells that are harvested from the subject being treated and/or biocompatible allogeneic or syngeneic cells, such as autologous, allogeneic, or syngeneic stem cells (e.g., mesenchymal stem cells), progenitor cells (e.g., connective tissue progenitor cells or multipotent adult progenitor cells) and/or other cells that are further differentiated and are biocompatible with the skeletal tissue being treated. The cells can include cells that are provided in bone grafts, engineered musculoskeletal tissue, and other musculoskeletal tissue replacement therapies that are used to treat skeletal injuries.

The agent can comprise natural or synthetic nucleic acids that are incorporated into recombinant nucleic acid constructs, typically DNA constructs, capable of introduction into and replication in the cell. Such a construct can include a replication system and sequences that are capable of transcription and translation of a polypeptide-encoding sequence in a given target cell.

Other agents can also be introduced into the cells to promote expression of SDF-1 and/or MCP-3 from the target cells. For example, agents that increase the transcription of a gene encoding SDF-1 or MCP-3, increase the translation of an mRNA encoding SDF-1 or MCP-3, and/or those that decrease the degradation of an mRNA encoding SDF-1 or MCP-3 could be used to increase SDF-1 or MCP-3 protein levels, respectively. Increasing the rate of transcription from a gene within a cell can be accomplished by introducing an exogenous promoter upstream of the gene encoding SDF-1 or MCP-3. Enhancer elements, which facilitate expression of a heterologous gene, may also be employed.

Other agents can further include other proteins, chemokines, and cytokines, that when administered to the target cells can upregulate expression SDF-1 and/or MCP-3 from the target cells. Such agents can include, for example: insulin-like growth factor (IGF)-1, which was shown to upregulate expression of SDF-1 when administered to mesenchymal stem cells (MSCs) (Circ. Res. 2008, Nov. 21; 103(11):1300-98); sonic hedgehog (Shh), which was shown to upregulate expression of SDF-1 when administered to adult fibroblasts (Nature Medicine, Volume 11, Number 11, Nov. 23); transforming growth factor β (TGF-β); which was shown to upregulate expression of SDF-1 when administered to human peritoneal mesothelial cells (HPMCs); IL-1β, PDG-BF, VEGF, TNF-a, and PTH, which are shown to upregulate expression of SDF-1, when administered to primary human osteoblasts (HOBs) mixed marrow stromal cells (BMSCs), and human osteoblast-like cell lines (Bone, 2006, April; 38(4): 497-508); thymosin P4, which was shown to upregulate expression when administered to bone marrow cells (BMCs) (Curr. Pharm. Des. 2007; 13(31):3245-51; and hypoxia inducible factor 1α (HIF-1), which was shown to upregulate expression of SDF-1 when administered to bone marrow derived progenitor cells (Cardiovasc. Res. 2008, E. Pub.). These agents can be used to promote musculoskeletal repair and treat specific musculoskeletal injuries where such cells capable of upregulating expression of SDF-1 and/or MCP-3 with respect to the specific cytokine are present or administered.

One method of introducing the agent into a target cell involves using gene therapy. Gene therapy in accordance with the present invention can be used to express SDF-1 protein and/or MCP-3 protein from a target cell in vivo or in vitro.

In an aspect of the application, the gene therapy can use a vector including a nucleotide encoding an SDF-1 protein and/or an MCP-3 protein. The SDF-1 nucleic acid and MCP-3 nucleic acid that encodes the SDF-1 protein and MCP-3 protein respectively, can be a native or non-native nucleic acid and be in the form of RNA or in the form of DNA (e.g., cDNA, genomic DNA, and synthetic DNA). The DNA can be double-stranded or single-stranded, and if single-stranded may be the coding (sense) strand or non-coding (anti-sense) strand.

The nucleic acid coding sequence that encodes SDF-1 may be substantially similar to a nucleotide sequence of the SDF-1 gene, such as nucleotide sequence shown in SEQ ID NO: 3 and SEQ ID NO: 4. SEQ ID NO: 3 and SEQ ID NO: 4 comprise, respectively, the nucleic acid sequences for human SDF-1 and rat SDF-1 and are substantially similar to the nucleic sequences of GenBank Accession No. NM199168 and GenBank Accession No. AF189724. The nucleic acid coding sequence for SDF-1 can also be a different coding sequence which, as a result of the redundancy or degeneracy of the genetic code, encodes the same polypeptide as SEQ ID NO: 1, and SEQ ID NO: 2.

Nucleic acid molecules that encode the MCP-3 can have sequences substantially similar to, respectively, SEQ ID NO: 6. SEQ ID NO: 6 is substantially similar to the nucleic sequences of GenBank Accession No. NM006273.

A “vector” (sometimes referred to as gene delivery or gene transfer “vehicle”) refers to a macromolecule or complex of molecules comprising a polynucleotide to be delivered to a target cell, either in vitro or in vivo. The polynucleotide to be delivered may comprise a coding sequence of interest in gene therapy. Vectors include, for example, viral vectors (such as adenoviruses (‘Ad’), adeno-associated viruses (AAV), and retroviruses), liposomes and other lipid-containing complexes, and other macromolecular complexes capable of mediating delivery of a polynucleotide to a target cell.

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities.

Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., Lupton, S., WO 92/08796, published May 29, 1992; and Lupton, S., WO 94/28143, published Dec. 8, 1994). Such marker genes can provide an added measure of control that can be advantageous in gene therapy contexts. A large variety of such vectors are known in the art and are generally available.

Vectors for use in the present invention include viral vectors, lipid based vectors and other non-viral vectors that are capable of delivering a nucleotide according to the present invention to the target cells. The vector can be a targeted vector, especially a targeted vector that preferentially binds to cells of the ischemic tissue. Viral vectors for use in the invention can include those that exhibit low toxicity to a target cell and induce production of therapeutically useful quantities of SDF-1 and/or MCP-3 protein in a tissue-specific manner.

Examples of viral vectors are those derived from adenovirus (Ad) or adeno-associated virus (AAV). Both human and non-human viral vectors can be used and the recombinant viral vector can be replication-defective in humans. Where the vector is an adenovirus, the vector can comprise a polynucleotide having a promoter operably linked to a gene encoding the SDF-1 protein and/or a gene encoding the MCP-3 protein and is replication-defective in humans.

Other viral vectors that can be used in accordance with the present invention include herpes simplex virus (HSV)-based vectors. HSV vectors deleted of one or more immediate early genes (IE) are advantageous because they are generally non-cytotoxic, persist in a state similar to latency in the target cell, and afford efficient target cell transduction. Recombinant HSV vectors can incorporate approximately 30 kb of heterologous nucleic acid.

Retroviruses, such as C-type retroviruses and lentiviruses, might also be used in the invention. For example, retroviral vectors may be based on murine leukemia virus (MLV). See, e.g., Hu and Pathak, Pharmacol. Rev. 52:493-511, 2000 and Fong et al., Crit. Rev. Ther. Drug Carrier Syst. 17:1-60, 2000. MLV-based vectors may contain up to 8 kb of heterologous (therapeutic) DNA in place of the viral genes. The heterologous DNA may include a tissue-specific promoter and an SDF-1 nucleic acid. The heterologous DNA may also include a tissue-specific promoter and a MCP-3 nucleic acid. The heterologous DNA may also include a heterologous DNA may include a tissue-specific promoter, an SDF-1 nucleic acid and a MCP-3 nucleic acid.

Additional retroviral vectors that might be used are replication-defective lentivirus-based vectors, including human immunodeficiency (HIV)-based vectors. See, e.g., Vigna and Naldini, J. Gene Med. 5:308-316, 2000 and Miyoshi et al., J. Virol. 72:8150-8157, 1998. Lentiviral vectors are advantageous in that they are capable of infecting both actively dividing and non-dividing cells.

Lentiviral vectors for use in the invention may be derived from human and non-human (including SIV) lentiviruses. Examples of lentiviral vectors include nucleic acid sequences required for vector propagation as well as a tissue-specific promoter operably linked to a SDF-1 gene. These former may include the viral LTRs, a primer binding site, a polypurine tract, att sites, and an encapsidation site.

A lentiviral vector may be packaged into any suitable lentiviral capsid. The substitution of one particle protein with another from a different virus is referred to as “pseudotyping”. The vector capsid may contain viral envelope proteins from other viruses, including murine leukemia virus (MLV) or vesicular stomatitis virus (VSV). The use of the VSV G-protein yields a high vector titer and results in greater stability of the vector virus particles.

In one particular example, stable transfection of a target cell is accomplished using a replication defective lentivirus encoding SDF-1 and MCP-3 (e.g., a bicistronic lentiviral vector). Cells with stable letiviral integration are selected in Blasticidin Selection media and expanded at 5% O₂ to passage number 9-12. Approximately 6×10⁵ cells were concentrated in 10 ul of media and pipette onto a 1×1×4 mm collagen-based bone substitute, HEALOS II (Depuy Inc, Raynham, Mass.). Alphavirus-based vectors, such as those made from semliki forest virus (SFV) and sindbis virus (SIN), might also be used in the invention. Use of alphaviruses is described in Lundstrom, K., Intervirology 43:247-257, 2000 and Perri et al., Journal of Virology 74:9802-9807, 2000.

Recombinant, replication-defective alphavirus vectors are advantageous because they are capable of high-level heterologous (therapeutic) gene expression, and can infect a wide target cell range. Alphavirus replicons may be targeted to specific cell types by displaying on their virion surface a functional heterologous ligand or binding domain that would allow selective binding to target cells expressing a cognate binding partner. Alphavirus replicons may establish latency, and therefore long-term heterologous nucleic acid expression in a target cell. The replicons may also exhibit transient heterologous nucleic acid expression in the target cell.

In many of the viral vectors compatible with methods of the invention, more than one promoter can be included in the vector to allow more than one heterologous gene to be expressed by the vector. Further, the vector can comprise a sequence which encodes a signal peptide or other moiety which facilitates the secretion of a SDF-1 gene product and/or a MCP-3 gene product from the target cell.

To combine advantageous properties of two viral vector systems, hybrid viral vectors may be used to deliver a SDF-1 nucleic acid and/or a MCP-3 nucleic acid to a target tissue. Standard techniques for the construction of hybrid vectors are well-known to those skilled in the art. Such techniques can be found, for example, in Sambrook, et al., In Molecular Cloning: A laboratory manual. Cold Spring Harbor, N.Y. or any number of laboratory manuals that discuss recombinant DNA technology. Double-stranded AAV genomes in adenoviral capsids containing a combination of AAV and adenoviral ITRs may be used to transduce cells. In another variation, an AAV vector may be placed into a “gutless”, “helper-dependent” or “high-capacity” adenoviral vector. Adenovirus/AAV hybrid vectors are discussed in Lieber et al., J. Virol. 73:9314-9324, 1999. Retrovirus/adenovirus hybrid vectors are discussed in Zheng et al., Nature Biotechnol. 18:176-186, 2000. Retroviral genomes contained within an adenovirus may integrate within the target cell genome and effect stable SDF-1 gene and/or MCP-3 gene expression.

Other nucleotide sequence elements which facilitate expression of SDF-1 gene and/or MCP-3 gene and cloning of the vector are further contemplated. For example, the presence of enhancers upstream of the promoter or terminators downstream of a coding region, for example, can facilitate expression.

In accordance with another aspect of the present invention, a tissue-specific promoter, can be fused to a SDF-1 gene and/or a MCP-3 gene. By fusing such tissue specific promoter within the adenoviral construct, transgene expression is limited to a particular tissue. The efficacy of gene expression and degree of specificity provided by tissue specific promoters can be determined, using the recombinant adenoviral system of the present invention.

In addition to viral vector-based methods, non-viral methods may also be used to introduce a SDF-1 nucleic acid and/or MCP-3 nucleic acid into a target cell. A review of non-viral methods of gene delivery is provided in Nishikawa and Huang, Human Gene Ther. 12:861-870, 2001. An example of a non-viral gene delivery method according to the invention employs plasmid DNA to introduce a SDF-1 nucleic acid and/or MCP-3 nucleic acid into a cell. Plasmid-based gene delivery methods are generally known in the art.

Synthetic gene transfer molecules can be designed to form multimolecular aggregates with plasmid DNA. These aggregates can be designed to bind to a target cell. Cationic amphiphiles, including lipopolyamines and cationic lipids, may be used to provide receptor-independent SDF-1 nucleic acid and/or MCP-3 nucleic acid transfer into target cells. In addition, preformed cationic liposomes or cationic lipids may be mixed with plasmid DNA to generate cell-transfecting complexes. Methods involving cationic lipid formulations are reviewed in Feigner et al., Ann N.Y. Acad. Sci. 772:126-139, 1995 and Lasic and Templeton, Adv. Drug Delivery Rev. 20:221-266, 1996. For gene delivery, DNA may also be coupled to an amphipathic cationic peptide (Fominaya et al., J. Gene Med. 2:455-464, 2000).

Methods that involve both viral and non-viral based components may be used according to the invention. For example, an Epstein Barr virus (EBV)-based plasmid for therapeutic gene delivery is described in Cui et al., Gene Therapy 8:1508-1513, 2001. Additionally, a method involving a DNA/ligand/polycationic adjunct coupled to an adenovirus is described in Curiel, D. T., Nat. Immun 13:141-164, 1994.

Additionally, the SDF-1 nucleic acid and/or MCP-3 nucleic acid can be introduced into the target cell by transfecting the target cells using electroporation techniques. Electroporation techniques are well known and can be used to facilitate transfection of cells using plasmid DNA.

Vectors that encode the expression of SDF-1 and/or MCP-3 nucleic acid can be delivered to the target cell in the form of an injectable preparation containing pharmaceutically acceptable carrier, such as saline, as necessary. Other pharmaceutical carriers, formulations and dosages can also be used in accordance with the present invention.

Where the target cell comprises a cell proximate the musculoskeletal injury being treated, the vector can be delivered by direct injection at an amount sufficient for the SDF-1 protein and/or MCP-3 protein to be expressed to a degree which allows for highly effective therapy. By injecting the vector directly into or about the periphery of the injury, it is possible to target the vector transfection rather effectively, and to minimize loss of the recombinant vectors. This type of injection enables local transfection of a desired number of cells, especially about the injury, thereby maximizing therapeutic efficacy of gene transfer, and minimizing the possibility of an inflammatory response to viral proteins.

Where the target cell is a cultured cell (e.g., mesenchymal stem cell) that is later transplanted into a musculoskeletal injury site, the vectors can be delivered by direct injection into the culture medium. A SDF-1 nucleic acid and/or MCP-3 nucleic acid transfected into cells may be operably linked to a regulatory sequence.

The transfected target cells can then be transplanted to the musculoskeletal injury by well known transplantation techniques, such as graft transplantation. By first transfecting the target cells in vitro and then transplanting the transfected target cells to the injury, the possibility of inflammatory response in the surrounding tissue is minimized compared to direct injection of the vector into cells proximate the injury.

SDF-1 and/or MCP-3 can be expressed for any suitable length of time within the target cell, including transient expression and stable, long-term expression. In one aspect of the invention, the SDF-1 nucleic acid and/or MCP-3 nucleic acid will be expressed in therapeutic amounts for a defined length of time effective to promote connective tissue progenitor cells homing to the injury and promote the healing of the musculoskeletal injury.

In an aspect of the application, the SDF-1 and/or MCP-3 can be administered to the musculoskeletal injury neat or in a therapeutic composition or pharmaceutical composition at a therapeutically effective amount. A therapeutically effective amount is an amount, which is capable of producing a medically desirable result in a treated animal or human. As is well known in the medical arts, dosage for any one animal or human depends on many factors, including the subject's size, body surface area, age, the particular composition to be administered, sex, time and route of administration, general health, and other drugs being administered concurrently. Specific dosages of proteins and nucleic acids can be determined readily determined by one skilled in the art using the experimental methods described below.

The pharmaceutical composition can provide localized release of the SDF-1 and/or MCP-3 to the area proximate the musculoskeletal injury or cells being treated. Pharmaceutical compositions in accordance with the invention will generally include an amount of SDF-1 and/or MCP-3 and variants thereof admixed with an acceptable pharmaceutical diluent or excipient, such as a sterile aqueous solution, to give a range of final concentrations, depending on the intended use. The techniques of preparation are generally well known in the art as exemplified by Remington's Pharmaceutical Sciences, 16th Ed. Mack Publishing Company, 1980, incorporated herein by reference. Moreover, for human administration, preparations should meet sterility, pyrogenicity, general safety and purity standards as required by FDA Office of Biological Standards.

The pharmaceutical composition can be in a unit dosage injectable form (e.g., solution, suspension, and/or emulsion). Examples of pharmaceutical formulations that can be used for injection include sterile aqueous solutions or dispersions and sterile powders for reconstitution into sterile injectable solutions or dispersions. The carrier can be a solvent or dispersing medium containing, for example, water, ethanol, polyol (e.g., glycerol, propylene glycol, liquid polyethylene glycol, and the like), suitable mixtures thereof and vegetable oils.

Proper fluidity can be maintained, for example, by the use of a coating, such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions

Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. However, any vehicle, diluent, or additive used would have to be compatible with the compounds.

Sterile injectable solutions can be prepared by incorporating the compounds utilized in practicing the present invention in the required amount of the appropriate solvent with various amounts of the other ingredients, as desired.

Pharmaceutical “slow release” capsules or “sustained release” compositions or preparations may be used and are generally applicable. Slow release formulations are generally designed to give a constant drug level over an extended period and may be used to deliver the SDF-1 and/or MCP-3. The slow release formulations are typically implanted in the vicinity of the skeletal injury site, for example, at the site in or about a skeletal fracture.

Examples of sustained-release preparations include semipermeable matrices of solid hydrophobic polymers containing the SDF-1 and/or MCP-3, which matrices are in the form of shaped articles, e.g., films or microcapsule. Examples of sustained-release matrices include polyesters; hydrogels, for example, poly(2-hydroxyethyl-methacrylate) or poly(vinylalcohol); polylactides, e.g., U.S. Pat. No. 3,773,919; copolymers of L-glutamic acid and γ ethyl-L glutamate; non-degradable ethylene-vinyl acetate; degradable lactic acid-glycolic acid copolymers, such as the LUPRON DEPOT (injectable microspheres composed of lactic acid glycolic acid copolymer and leuprolide acetate); and poly-D-(−)-3-hydroxybutyric acid.

While polymers such as ethylene-vinyl acetate and lactic acid-glycolic acid enable release of molecules for over 100 days, certain hydrogels release proteins for shorter time periods. When encapsulated SDF-1 and/or MCP-3 remain in the body for a long time, and may denature or aggregate as a result of exposure to moisture at 37° C., thus reducing biological activity and/or changing immunogenicity. Rational strategies are available for stabilization depending on the mechanism involved. For example, if the aggregation mechanism involves intermolecular S—S bond formation through thio-disulfide interchange, stabilization is achieved by modifying sulfhydryl residues, lyophilizing from acidic solutions, controlling moisture content, using appropriate additives, developing specific polymer matrix compositions, and the like.

In certain embodiments, liposomes and/or nanoparticles may also be employed with the SDF-1 and/or MCP-3. The formation and use of liposomes is generally known to those of skill in the art, as summarized below.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Phospholipids can form a variety of structures other than liposomes when dispersed in water, depending on the molar ratio of lipid to water. At low ratios, the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via four different mechanisms: Endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one may operate at the same time.

Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and such particles may be are easily made.

For preparing pharmaceutical compositions from the signaling molecules of the present invention, pharmaceutically acceptable carriers can be in any suitable form (e.g., solids, liquids, gels, etc.). A solid carrier can be one or more substances which may also act as diluents, binders, preservatives, and/or an encapsulating material.

The signaling molecules (e.g., SDF-1 and MCP-3 and variants thereof) described herein can be provided in and/or on a substrate, solid support, and/or wound dressing for delivery of at least one signaling molecule to the wound. As used herein, the term “substrate,” or “solid support” and “wound dressing” refer broadly to any substrate when prepared for, and applied to, a wound for protection, absorbance, drainage, etc. The present invention may include any one of the numerous types of substrates and/or backings that are commercially available, including films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates (non-woven composites of fibers from calcium alginate), and cellophane (cellulose with a plasticizer). The shape and size of a wound may be determined and the wound dressing customized for the exact site based on the measurements provided for the wound. As musculoskeletal injury sites can vary in terms of mechanical strength, thickness, sensitivity, etc., the substrate can be molded to specifically address the mechanical and/or other needs of the site.

In one example, the substrate can be a bioresorbable implant that includes a polymeric matrix and the SDF-1 and/or MCP-3 (or cells expressing SDF-1 and/or MCP-3, or vectors that can be used to express SDF-1 and/or MCP-3) dispersed in the matrix. The polymeric matrix may be in the form of a membrane, sponge, gel, or any other desirable configuration. The polymeric matrix can be formed from biodegradable polymer. It will be appreciated, however, that the polymeric matrix may additionally comprise an inorganic or organic composite. The polymeric matrix can comprise anyone or combination of known materials including, for example, chitosan, poly(ethylene oxide), poly (lactic acid), poly(acrylic acid), poly(vinyl alcohol), poly(urethane), poly(N-isopropyl acrylamide), poly(vinyl pyrrolidone) (PVP), poly (methacrylic acid), poly(p-styrene carboxylic acid), poly(p-styrenesulfonic acid), poly(vinylsulfonicacid), poly(ethyleneimine), poly(vinylamine), poly(anhydride), poly(Llysine), poly(L-glutamic acid), poly(gamma-glutamic acid), poly(carprolactone), polylactide, poly(ethylene), poly(propylene), poly(glycolide), poly(lactide-co-glycolide), poly(amide), poly(hydroxylacid), poly(sulfone), poly(amine), poly(saccharide), poly(HEMA), poly(anhydride), collagen, gelatin, glycosaminoglycans (GAG), poly (hyaluronic acid), poly(sodium alginate), alginate, hyaluronan, agarose, polyhydroxybutyrate (PHB), and the like.

It will be appreciated that one having ordinary skill in the art may create a polymeric matrix of any desirable configuration, structure, or density. By varying polymer concentration, solvent concentration, heating temperature, reaction time, and other parameters, for example, one having ordinary skill in the art can create a polymeric matrix with any desired physical characteristic(s). For example, the polymeric matrix may be formed into a sponge-like structure of various densities. The polymeric matrix may also be formed into a membrane or sheet, which could then be wrapped around or otherwise shaped to a wound. The polymeric matrix may also be configured as a gel, mesh, plate, screw, plug, or rod. Any conceivable shape or form of the polymeric matrix is within the scope of the present invention. In an example of the present invention, the polymeric matrix can comprise an osteoconductive matrix.

In another aspect of the invention, the polymeric matrix may be seeded with a population of mammalian cells expressing or promoting expression of SDF-1 and/or MCP-3. The SDF-1 and/or MCP-3 expressing cell population can be dispersed in the matrix. Mammalian cells can include autologous cells; however, it will be appreciated that xenogeneic, allogeneic, or syngeneic cells may also be used. Where the cells are not autologous, it may be desirable to administer immunosuppressive agents in order to minimize immunorejection. The progenitor cells employed may be primary cells, explants, or cell lines, and may be dividing or non-dividing cells. The mammalian cells may be expanded ex vivo prior to introduction into the polymeric matrix. Autologous cells are preferably expanded in this way if a sufficient number of viable cells cannot be harvested from the host. In certain aspects of the present invention, the mammalian cell population includes mesenchymal stem cells.

In other aspects, the polymer matrix seeded with the population of mammalian cells expressing or promoting expression of SDF-1 and/or MCP-3 can comprise bone graft or bone graft substitute. In some embodiments, an osteoconductive matrix can be used to support the mammalian cells and include collagen fibers coated with hydroyapatite. In other aspects, the osteoconductive matrix is saturated with the population of cells. In one particular example, SDF-1 and/or MCP-3 is delivered to the musculoskeletal injury or to an area proximate the skeletal injury by syngenic culture-expanded mesenchymal stem cells. The seeded osteoconductive matrix may then be implanted adjacent to a bone fracture site for the treatment of a skeletal injury in a subject.

In another aspect of the application, a therapeutic composition can include a bone graft, such as an autograft, that seeded with SDF-1 and/or MCP-3, or includes cells that are genetically modified to upregulate expression of SDF-1 and/or MCP-3. An autograft bone includes a portion of bone that is removed from a subject's body when a grafting procedure is necessary. Bone grafting is commonly used to repair fractured bones. While grafting can include artificial bone replacement, autografting is often the most successful type of grafting available. Bones tend to more readily adhere to one another when a subject's own bone is used. The most common donor area is the iliac crest, which is located in the subject's pelvis.

A bone graft may also include an allograft bone graft. Typically, an allograft bone graft is bone obtained from cadavers. An allograft may be sterilized and/or fresh frozen or freeze-dried prior to grafting. An allograft may also be used as a bone graft supplement (to the subject's own bone) in subjects.

In a further aspect, the SDF-1 and/or MCP-3 can be provided in or on a surface of a medical device used to treat a musculoskeletal injury. The medical device can comprise any instrument, implement, machine, contrivance, implant, or other similar or related article, including a component or part, or accessory which is recognized in the official U.S. National Formulary, the U.S. Pharmacopoeia, or any supplement thereof; is intended for use in the diagnosis of disease or other conditions, or in the cure, mitigation, treatment, or prevention of disease, in humans or in other animals; or, is intended to affect the structure or any function of the body of humans or other animals, and which does not achieve any of its primary intended purposes through chemical action within or on the body of man or other animals, and which is not dependent upon being metabolized for the achievement of any of its primary intended purposes.

It is to be noted that throughout this application various publications and patents are cited. The disclosures of these publications are hereby incorporated by reference in their entireties into this application in order to describe fully the state of the art to which this invention pertains.

The invention will be further described in the following examples, which do not limit the scope of the invention described in the claims.

### Example

This Example shows that SDF-1 and MCP-3 over expression can be used to enhance osteoblastic precursor recruitment following bone fracture.

### Materials and Methods

### Animal Model

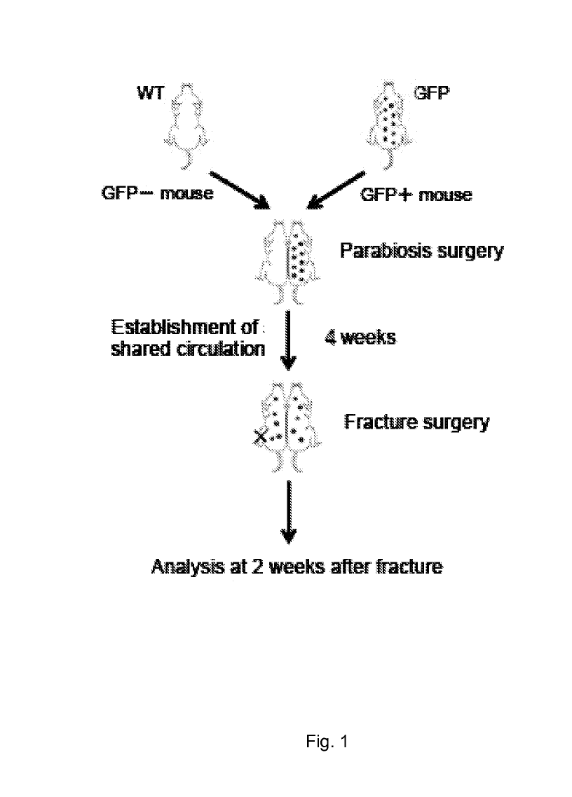

Fifty pairs of transgenic GFP+ and wild type (GFP−) C57BL/6 mice were surgically conjoined as parabiots at 7-8 weeks of age, as previously described. Anesthesia was obtained with intraperitoneal injection of sodium pentobarbital (0.6 mg per 10 g body weight), allowing for the dorsal and opposing lateral aspects of each mouse to be shaved and sterilized. Matching longitudinal skin incisions were made to allow for blunt dissection of the subcutaneous layer in order to create a free skin flap. The scapulas and paraspinal muscles were sutured together. The olecranon and knee joints were bound and the dorsal and ventral skin was sewn together using a continuous suture. Fibular osteotomy was performed 4 weeks after parabiosis on the left hind limb of the GFP— animal in each pair (FIG. 1A). All procedures were conducted in accordance with principles and procedures approved by the IACUC committee at Cleveland Clinic.

### Experimental Groups

To study the effects of homing factor, animals were divided into 5 groups (Table 1). Ten parabiosis mice were included in each group. To determine the effect of a collagen scaffold/cell delivery system on the natural expression, we then evaluated the effect of each individual.

TABLE 1
Experimental Group

| Group | Experimental |
|---|---|
| 1 | Fracture |
| 2 | Fracture + Scaffold |
| 3 | Fracture + Scaffold seeded with Mesenchymal Stem Cells |
| 4 | Fracture + Scaffold seeded with MSCs that express SDF-1 |
| 5 | Fracture + Scaffold seeded with MSCs that express MCP-3 |

### Bone Marrow Harvest and Culture

Delivery of secreted homing molecules was accomplished using syngeneic, culture-expanded MSCs isolated from the femur and tibia of 4-5 week old mice. After harvest of the femur and tibia and removal of proximal and distal epiphyses, 5 ml of growth medium was used to flush bone marrow from the medullary canal of both femur and tibia through a 70-micron cell strainer (BD Biosciences, San Jose, Calif.). Cells were spun at 2000 rpm for five minutes, after which the media was aspirated, leaving a cell pellet. Cells were resuspended in 5 ml phosphorous buffered saline (PBS) and centrifuged at 2000 rpm for five minutes. This process was repeated two additional times to thoroughly wash the cells. The final pellet was re-suspended in 12 ml of culture media and plated in a nonpyrogenic polystyrene T75 flask (Corning Inc, Lowell, Mass.). The flask was placed in a hypoxic incubator (oxygen tension 5%, temperature 37° C.). Cells were allowed to incubate until 70-80% confluence before splitting. To split a flask, the culture media was aspirated and the cells were gently washed with PBS and lifted by incubation with 0.05% trypsin/0.53 mM EDTA for two minutes. The trypsin was neutralized with addition of culture media. Cells were split roughly 1:4.

### MSC Selection and Viral Transduction

Cells were cultured until passage six, at which point they were immunodepleted of CD45 and CD34⁺ hematopoetic cells using EasySep magnetic cell sorting system (Stem Cell Technologies, Vancouver, BC, Canada). Antibodies used included FcR blocker, PE anti CD45, and PE anti CD34 (BD Biosciences, San Jose, Calif.). Remaining MSCs were transduced with 1×10⁵ transducing units (TU)/ml in the presence of polybrene (8 μg/ml). Successfully transduced cells were identified through Blasticidin resistance conferred by the expression plasmid. Cells were again expanded to obtain sufficient numbers for implantation and harvested between passages eight and ten.

### Lentivirus Preparation

Lentivirus stocks were produced by transient cotransfection of the construct pCCLsin.cPPT.hPGK.hSDF-1-IRES-eGFP.Wpre and the third-generation packaging constructs pMDL, pRSV-Rev and pTK-Rev in 293T cells followed by ultracentrifugation of conditioned medium. Endpoint expression titer in 293FT cells was used to calculate the titer of the lentivirus stock. The lentivirus delivery system contained either an SDF-1 or MCP-3 construct under a chick beta-actin promoter and CMV enhancer.

### Seeding of Healos Scaffold

Cells were lifted, separated, and re-suspended to achieve a cell concentration of 6×10⁵ cells per 20 μl of growth medium. A 1×1×4 mm section of HEALOS® (Depuy Inc, Raynham, Mass.) was placed within the 50 μl culture wells in Multi-Well (96) Tissue Culture Dish with Ultra Low Attachment Surface (Corning, Lowell, Mass.). The plates were held at a 45-degree angle to maximize the depth of cell solution as it was slowly pipetted onto the scaffold for incubation. At 30 minutes, the cells settled to the bottom were re-suspended in an additional 10 μl growth medium and pipetted onto the opposite side of the scaffold in order to ensure homogenous scaffold seeding. The cells were allowed to passively adhere to the scaffold for 3 hours in the standard hypoxic incubator, at which point they were transferred under sterile conditions to the surgical suite for implantation.

### Fracture Surgery

The animal was then draped in sterile fashion such that only the sterile hind limb was exposed. Traction was applied to the operative limb to aid with stability and help delineate muscle bellies in the lower limb. A five mm incision was made through the skin in the indentation between the anterior and posterior muscle compartments. A surgical microscope allowed adequate visualization as blunt dissection techniques were used to expose of the fibula. Fine tip dissection scissors were used to create a transverse fracture approximately 4 mm from the proximal origin. Depending on treatment group, a 1×1×4 mm HEALOS® Bone Graft Replacement was placed within the tissue pocket on the anterior surface of the fibula prior to osteotomy. In both scenarios, no effort to approximate the two ends of the fracture was made, although minimal displacement was noted intra-operatively in all cases.

### Histological Assessment

Mice were euthanized two weeks post osteotomy. The fracture region was harvested and stored at 4° C. during processing for cryosection. Samples were fixed in 4% formaldehyde overnight, decalcified in 0.5 mol/L ETDA in 1×PBS for 14 days (changed solution every other day), and dehydrated in 20% sucrose solution. Samples were embedded in embedding medium Tissue-Tek® O.C.T.™ Compound (Sakura Finetek U.S.A, Torrance, Calif.), frozen on dry ice and stored in the dark at −800 C until sectioned. Sagittal sections (15 μm) through the fracture callus were obtained. They were stained with Vector Red Alkaline Phosphatase Substrate solution (Vector Laboratories, Burlinggame, Calif.) and Vectashield mounting medium containing 4′-6′-diaminido-2-phenylindole (DAPI) (Vector Laboratories, Burlingame, Calif.) and stored in the dark at 40 C until imaged.

### Histomorphometrical Quantification

Imaging was performed using the Leica DMIRBE (TCS—SPAOBS) confocal laser scanning microscope (Leica Microsystems, Heidelberg, Germany). Three slides per animal were used for confocal imaging and 2 digital images per section with high power fields (40×) within callus near scaffold were obtained for cell counting analysis based on relative callus size and gross morphology. Regions of interest were scanned to obtain DAPI, AP, and GFP images. Emissions from DAPI, GFP and AP were detected with spectrophotometer at a range of 460-600 nm, 540-599 nm and 480-600 nm, respectively.

Using Image-Pro Plus software (Media Cybernetics, Silver Spring, Md.), a software macro was designed to determine the total cell count, the number of AP⁺, GFP⁺, and AP⁺/GFP⁺ cells in each overlay image. Cell number was determined by counting cell nuclei in DAPI images. Segmentation of nuclei in the DAPI channel was performed by applying spectral filters to equalize intensity of nuclei, a morphological “watershed” filter to separate touching nuclei, and size/shape thresholds to remove objects not consistent with the general appearance of a nucleus. AP⁺ cells were identified by “dilating” each nucleus in the binary mask generated in the prior step to create a donut shaped region (5 pixels thick) surrounding each nucleus. This resultant mask was then multiplied with the AP channel; if an AP pixel within a given dilated, nuclear region was higher than a minimum predefined threshold, the cell was classified as AP⁺. GFP⁺ cells were identified using the same dilated binary mask used. The GFP signal was reduced for each image by selecting a “non-positive” region of interest whose mean value was subtracted from the entire GFP channel. A median filter, which smoothed the image by assigning the median value of the nine neighboring pixels to the center pixel, was used to remove high intensity background pixels. If the average GFP signal within the binary mask was greater than a specified value, the cell was characterized as GFP⁺. Cells were then classified as GFP⁺/AP⁺, GFP⁺/AP⁻, GFP⁻/AP⁺, or GFP⁻/AP⁻. AP⁺ cells within the fracture callus were interpreted to be osteogenic cells (osteoblasts or pre-osteoblasts). GFP cells were interpreted to be the cells, or progeny of cells that arrived in the fracture callus from systemic circulation. Double positive cells (GFP⁺/AP⁺) were interpreted as osteogenic cells derived from cells recruited through systemic circulation. Cell density was determined by calculating the number of cells within the region of interest and converting values to cells/mm².

### Statistical Analysis

A mean value for each of the six variables listed above was determined for each mouse by averaging data from between one and six images per callus. Using SAS's JMP software, these variables where then compared between treatment groups using one-way ANOVA. An alpha value of 0.025 was used based on Bonferroni correction for multiple comparisons.

### Results

### Postoperative Status of Parabiosis Animals

All animals recovered on the day of parabiosis surgery and rapidly learned to move together, cooperating in walking, and taking turns when getting access to food and water postoperatively. Ten parabiosis pairs died during the experimental period. These animals were eliminated from analysis and were replaced.

### Histological Findings of Fracture Callus in Parabiosis Wild-Type Partner

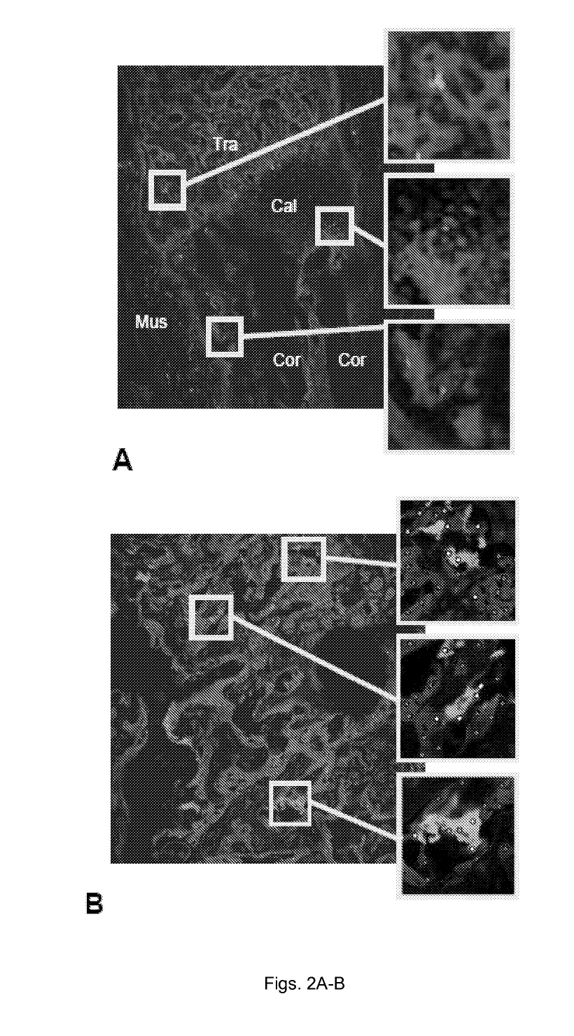

Two to four callus formation areas around the fracture region in each slide were examined. Formation of new soft cartilaginous callus was observed at fracture site and the surrounding area. Cartilage was partially resorbed and hard callus formation was evident. Compartments of cortical bone were connected by newly formed bone and the partial marrow space was repopulated with hematopoietic marrow (FIG. 2A).

GFP⁺ cells gaining access to the fracture site via systemic circulation were identified in all groups. More GFP⁺ cells were present in SDF-1 and MCP-3 groups compared to other groups. There was no difference in GFP+ expression in other groups. For DAPI and AP activity, no significant difference in all groups was demonstrated (FIG. 2B).

### Homing of the Circulating Cells to the Fracture Callus

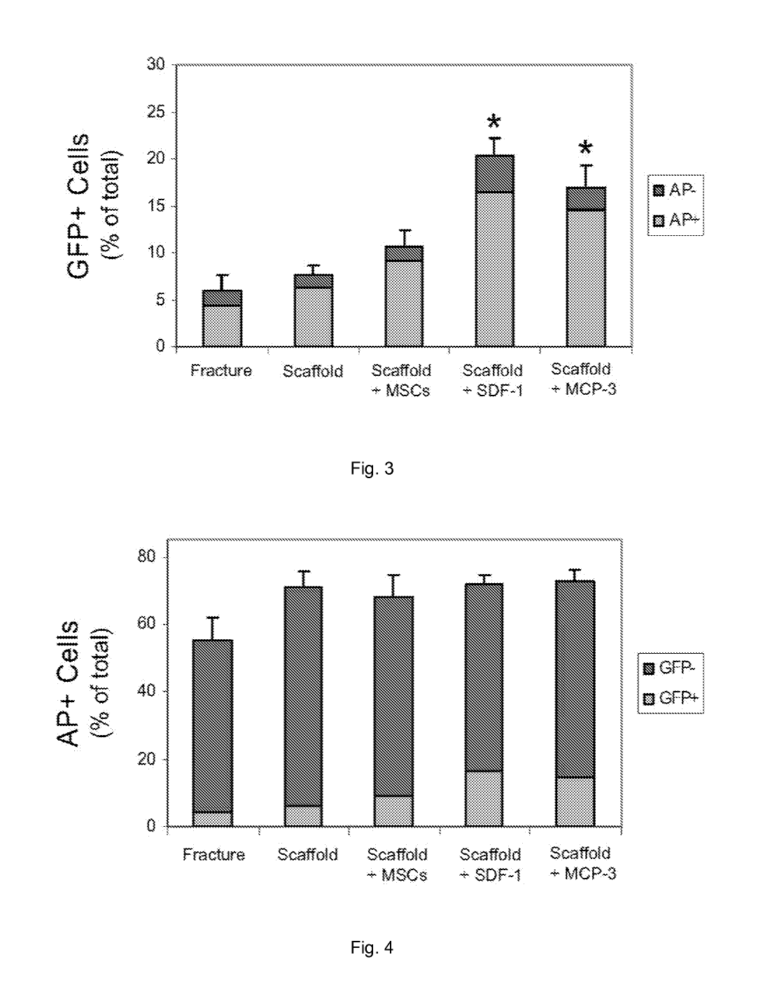

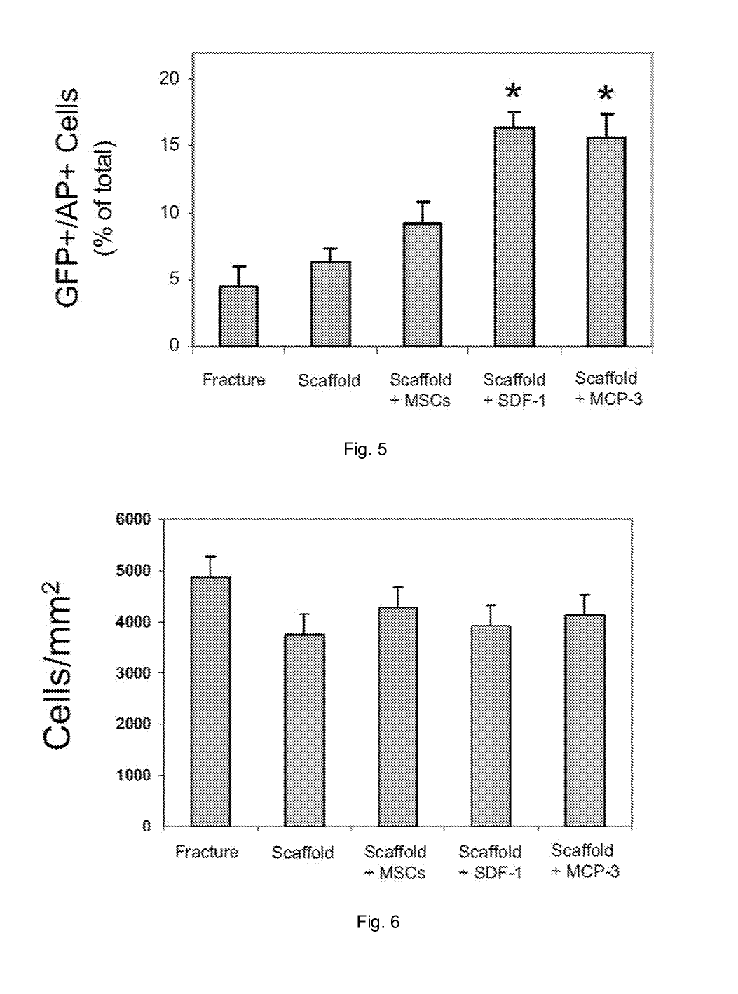

The percentage of GFP⁺ cells in the fracture region for Groups 1-5 was 5.8, 7.6, 10.7, 20.3 and 16.9, respectively. Both SDF-1 group and MCP-3 group demonstrated a statistically significant higher percentage of GFP⁺ cells compared to control group (FIG. 3A).

The percentage of AP⁺ cells in the fracture region in G1-5 was 55, 69, 70, 71 and 72, respectively. This was no significant difference between groups (FIG. 3B). The percentage of AP⁺ cells in control group (no scaffold), however, was relatively smaller than other groups.

To evaluate the possible contribution of GFP⁺ cells to osteogenesis, co-localization of GFP expression and AP activity was assessed for each group. Of the GFP⁺ cells present, the percentage of AP⁺ cells at Group 1-5 was 4.4, 6.3, 9.1, 14.5 and 16.4, respectively (FIG. 3C). Both SDF-1 and MCP-3 had a significantly higher percentage of cells expressing AP activity and GFP cell homing in callus bone at the fracture site (p<0.001). Post hoc multiple comparison test indicated that the change in percentage of AP⁺ and GFP⁺ cells was statistically significantly higher in both SDF-1 and MCP-3 groups (p<0.01) compared with either control group (no scaffold) or MSCs group. Additionally, there appeared to be no benefit from addition of scaffold or generic MSCs in recruitment of any cell type.

Overall, in all groups approximately 80% of all GFP⁺ cells in the fracture sites was AP⁺ and 15% of all AP⁺ cells was GFP⁺ (FIGS. 3A, B), with no significant difference in overall AP⁺ cells prevalence between groups. No difference in callus cellularity was demonstrated amongst all groups (FIG. 3D).

Expression of either SDF-1 or MCP-3 at the fracture site resulted in a significant increase in homing of connective tissue progenitor cells (CTPs) to a fracture site. Roughly 20% and 17% of the total cells within the fracture calluses of SDF-1 and MCP-3 treated calluses expressed GFP, respectively. MSCs treatment alone resulted in roughly 10% GFP⁺ cells, where as natural homing results in roughly 6% GFP+ cells. Assuming systemic cell recruitment was independent of cell origin (from which mouse the cell was derived), approximately 40% and 34% (given half of circulating cells are GFP⁺) of cells within the calluses were recruited through circulation. An identical trend of recruiting through circulation is seen regarding GFP⁺/AP⁺ cells. The implications of these findings are that both SDF-1 and MCP-3 expression can recruit CTPs that retain the capability of bone formation. We did not find a difference in either the percentage of cells within the callus that participated in bone formation or cellular density. We calculated cellular density as a method to assess (the early stages of fracture maturity. Decreased cellular density is seen with progression from a highly cellular soft callus to a relative matured remodeled callus at fracture site.

Parabiosis is a well established model to accomplish blood chimerism between partners, which allows investigation of physiological rate and fate of cells transiting through systemic circulation. We showed that homeostasis at a near equivalent level of blood chimerism was established by 2 weeks following parabiosis. This allowed a fracture to be created in the GFP− partner at 3 weeks postparabiosis surgery under conditions where comparable contribution of circulating cells could be expected from both GFP and GFP-recipient.

These data suggest a role of both of these signaling molecules as therapeutic modulators that can be used to enhance the homing of osteogenic progenitors into sites of bone repair.

While this invention has been shown and described with references to various embodiments thereof, it will be understood by those skilled in the art that changes in form and details may be made therein without departing from the scope of the invention encompassed by the appended claims. All patents, publications and references cited in the foregoing specification are herein incorporated by reference in their entirety.

Having described the invention, the following is claimed:

1. A method of treating a musculoskeletal injury in a subject, comprising:
administering directly to a site of the musculoskeletal injury or to an area proximate the musculoskeletal injury an amount of SDF-1, MCP-3, or combinations thereof effective to promote repair of the musculoskeletal injury of the subject and recruit connective tissue progenitor cells to the site of the musculoskeletal injury.

2. The method of claim 1, the SDF-1 and/or MCP-3 being administered by expressing or promoting expression of SDF-1 and/or MCP-3 from a cell or cells proximate the musculoskeletal injury.

3. The method of claim 2, the cell or cells proximate the musculoskeletal injury comprising a mesenchymal stem cell.

4. The method of claim 2, the cell or cells proximate the musculoskeletal injury being included in an osteoconductive matrix that is administered directly to the musculoskeletal injury.

5. The method of claim 4, the osteoconductive matrix being resorbed and remodeled into new bone as part of the natural healing process of the subject.

6. The method of claim 3, the cell or cells expressing the SDF-1 and/or MCP-3 being genetically modified by at least one of a vector, plasmid DNA, electroporation, and nano-particles to express SDF-1 and/or MCP-3.

7. The method of claim 1, the SDF-1 and/or MCP-3 being delivered in a local formulation that is administered directly to the site of the musculoskeletal injury or to an area proximate the musculoskeletal injury.

8. The method of claim 7, the local formulation comprising at least one of SDF-1 protein, an SDF-1 vector for expressing SDF-1 from cells of the musculoskeletal injury or proximate the musculoskeletal injury.

9. The method of claim 7, the local formulation comprising at least one of MCP-3 protein, an SDF-1 vector for expressing SDF-1 from cells of the musculoskeletal injury or proximate the musculoskeletal injury.

10. The method of claim 1, the musculoskeletal injury comprising a skeletal fracture.

11. A method of treating a musculoskeletal injury in a subject, comprising:
administering directly to a site of the musculoskeletal injury or to an area proximate the musculoskeletal injury an amount of SDF-1 and MCP-3 effective to promote repair of the musculoskeletal injury of the subject and recruit connective tissue progenitor cells to the site of the musculoskeletal injury.

12. The method of claim 11, the SDF-1 and MCP-3 being administered by expressing or promoting expression of SDF-1 and MCP-3 from a cell or cells proximate the musculoskeletal injury.

13. The method of claim 12, the cell or cells proximate the musculoskeletal injury comprising a mesenchymal stem cell.

14. The method of claim 12, the cell or cells proximate the musculoskeletal injury being included in an osteoconductive matrix that is administered directly to the musculoskeletal injury.

15. The method of claim 14, the osteoconductive matrix being resorbed and remodeled into new bone as part of the natural healing process of the subject.

16. The method of claim 12, the cell or cells expressing the SDF-1 and MCP-3 being genetically modified by at least one of a vector, plasmid DNA, electroporation, and nano-particles to express SDF-1 and MCP-3.

17. The method of claim 11, the SDF-1 and MCP-3 being delivered in a local formulation that is administered directly to the site of the musculoskeletal injury or to an area proximate the musculoskeletal injury.

18. The method of claim 11, the musculoskeletal injury comprising a skeletal fracture.

19. A bone graft or bone graft substitute for treating a musculoskeletal injury, the bone graft or bone graft substitute comprising:
an amount of SDF-1 and/or MCP-3 effective to promote repair of the musculoskeletal injury of the subject and recruit connective tissue progenitor cells to the site of the musculoskeletal injury; and
an osteoconductive matrix.

20. The bone graft or bone graft substitute of claim 19, further comprising a population of cells, the cells over expressing the amount of SDF-1 and/or MCP-3 effective to promote repair of the skeletal injury of the subject and recruit connective tissue progenitor cells to the site of the musculoskeletal injury.

21. The bone graft or bone graft substitute of claim 20, the population of cells comprising mesenchymal stem cells.

22. The bone graft or bone graft substitute of claim 20, the cells being autologous to the subject being treated.

23. The bone graft or bone graft substitute of claim 19, the osteoconductive matrix comprising collagen fibers coated with hydroyapatite.

24. The bone graft or bone graft substitute of claim 19, the osteoconductive matrix being saturated with the population of cells.

25. The bone graft or bone graft substitute of claim 19, further at least one of a vector, plasmid DNA, and nano-particles that can transfect a cell in the graft or proximate the graft to express SDF-1 and/or MCP-3.

26. The bone graft or bone graft substitute of claim 19, further comprising SDF-1 protein and/or MCP-3 protein interspersed in the osteoconductive matrix.


SEQUENCE LISTING (6 sequences)

SEQ ID NO: 1 - protein, 89 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNK

SEQ ID NO: 2 - protein, 89 aa, Rattus norvegicus
MDAKVVAVLALVLAALCISDGKPVSLSYRCPCRFFESHVARANVKHLKILNTPNCALQIVARLKSNNRQVCIDPKLKWIQEYLDKALNK

SEQ ID NO: 3 - DNA, 1940 bp, Homo sapiens
gccgcactttcactctccgtcagccgcattgcccgctcggcgtccggcccccgacccgcgctcgtccgcccgcccgcccgcccgcccgcgccatgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagtaagcacaacagccaaaaaggactttccgctagacccactcgaggaaaactaaaaccttgtgagagatgaaagggcaaagacgtgggggagggggccttaaccatgaggaccaggtgtgtgtgtggggtgggcacattgatctgggatcgggcctgaggtttgccagcatttagaccctgcatttatagcatacggtatgatattgcagcttatattcatccatgccctgtacctgtgcacgttggaacttttattactggggtttttctaagaaagaaattgtattatcaacagcattttcaagcagttagttccttcatgatcatcacaatcatcatcattctcattctcattttttaaatcaacgagtacttcaagatctgaatttggcttgtttggagcatctcctctgctcccctggggagtctgggcacagtcaggtggtggcttaacagggagctggaaaaagtgtcctttcttcagacactgaggctcccgcagcagcgcccctcccaagaggaaggcctctgtggcactcagataccgactggggctgggcgccgccactgccttcacctcctctttcaacctcagtgattggctctgtgggctccatgtagaagccactattactgggactgtgctcagagacccctctcccagctattcctactctctccccgactccgagagcatgcttaatcttgcttctgcttctcatttctgtagcctgatcagcgccgcaccagccgggaagagggtgattgctggggctcgtgccctgcatccctctcctcccagggcctgccccacagctcgggccctctgtgagatccgtctttggcctcctccagaatggagctggccctctcctggggatgtgtaatggtccccctgcttacccgcaaaagacaagtctttacagaatcaaatgcaattttaaatctgagagctcgctttgagtgactgggttttgtgattgcctctgaagcctatgtatgccatggaggcactaacaaactctgaggtttccgaaatcagaagcgaaaaaatcagtgaataaaccatcatcttgccactaccccctcctgaagccacagcagggtttcaggttccaatcagaactgttggcaaggtgacatttccatgcataaatgcgatccacagaaggtcctggtggtatttgtaactttttgcaaggcatttttttatatatatttttgtgcacatttttttttacgtttctttagaaaacaaatgtatttcaaaatatatttatagtcgaacaattcatatatttgaagtggagccatatgaatgtcagtagtttatacttctctattatctcaaactactggcaatttgtaaagaaatatatatgatatataaatgtgattgcagcttttcaatgttagccacagtgtattttttcacttgtactaaaattgtatcaaatgtgacattatatgcactagcaataaaatgctaattgtttcatggtataaacgtcctactgtatgtgggaatttatttacctgaaataaaattcattagttgttagtgatggagcttaaaaaaaa

SEQ ID NO: 4 - DNA, 293 bp, Rattus norvegicus
ccatggacgccaaggtcgtcgctgtgctggccctggtgctggccgcgctctgcatcagtgacggtaagccagtcagcctgagctacagatgcccctgccgattctttgagagccatgtcgccagagccaacgtcaaacatctgaaaatcctcaacactccaaactgtgcccttcagattgttgcaaggctgaaaagcaacaacagacaagtgtgcattgacccgaaattaaagtggatccaagagtacctggacaaagccttaaacaagtaagcacaacagcccaaaggactt

SEQ ID NO: 5 - protein, 99 aa, Homo sapiens
MKASAALLCLLLTAAAFSPQGLAQPVGINTSTTCCYRFINKKIPKQRLESYRRTTSSHCPREAVIFKTKLDKEICADPTQKWVQDFMKHLDKKTQTPKL

SEQ ID NO: 6 - DNA, 810 bp, Homo sapiens
agcagaggggctgagaccaaaccagaaacctccaattctcatgtggaagcccatgccctcaccctccaacatgaaagcctctgcagcacttctgtgtctgctgctcacagcagctgctttcagcccccaggggcttgctcagccagttgggattaatacttcaactacctgctgctacagatttatcaataagaaaatccctaagcagaggctggagagctacagaaggaccaccagtagccactgtccccgggaagctgtaatcttcaagaccaaactggacaaggagatctgtgctgaccccacacagaagtgggtccaggactttatgaagcacctggacaagaaaacccaaactccaaagctttgaacattcatgactgaactaaaaacaagccatgacttgagaaacaaataatttgtataccctgtcctttctcagagtggttctgagattattttaatctaattctaaggaatatgagctttatgtaataatgtgaatcatggtttttcttagtagattttaaaagttattaatattttaatttaatcttccatggattttggtgggttttgaacataaagccttggatgtatatgtcatctcagtgctgtaaaaactgtgggatgctcctcccttctctacctcatgggggtattgtataagtccttgcaagaatcagtgcaaagatttgctttaattgttaagatatgatgtccctatggaagcatattgttattatataattacatatttgcatatgtatgactcccaaattttcacataaaatagatttttgtaaaaaa